SEQUENCE LISTING (15 sequences)

SEQ ID NO: 1 - DNA, 63 bp, Artificial Sequence
  signal sequence
gccatgagcaaagatggtaaaaagaagaaaaagaagtcaaagacaaagtgtgtaattatgtaa

SEQ ID NO: 2 - protein, 384 aa, Homo sapiens
KLARHSKFGMKDFSWFGFGKVKSRQGVGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 3 - DNA, 1152 bp, Homo sapiens
aagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggc

SEQ ID NO: 4 - DNA, 1263 bp, Artificial Sequence
  nucleic acid sequence coding for CA-TrkB
atggcatcaatgcagaagctgatctcagaggaggacctgaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcgcggccgcggccatgagcaaagatggtaaaaagaagaaaaagaagtcaaagacaaagtgtgtaattatgtaa

SEQ ID NO: 5 - DNA, 1851 bp, Artificial Sequence
  nucleic acid sequence coding for iSH-TrkB
atggcatcaatgcagaagctgatctcagaggaggacctggtgtccaaataccaacaggatcaagttgtcaaagaagataatattgaagctgtagggaaaaaattacatgaatataacactcaatttcaagaaaaaagtcgggaatatgatagattatatgaagactatacccgaacttcccaggaaatccaaatgaaaagaacagctattgaagcatttaatgaaaccataaaaatatttgaagaacagtgccaaacccaggagcggtacagcaaagaatacatagaaaagttcaaacgtgaaggcaacgagacggaaatccaaaggattatgcataattatgaaaagttaaagtctcgaatcagtgaaattgtcgatagcagaaggagattggaagaggacttgaagaagcaggcggctgagtatcgtgagattgacaaacgcatgaacagcattaaaccagacctcattcagctaagaaagacgagagaccaatacttgatgtggttgactcagaaaggtgttcggcagaagaaattgaacgagtggctgggcaatgaaaacactgaagaccaatattcgctggtagaagatgatgaggatctgccccaccacgatgagaagactaagttggcaagacactccaagtttggcatgaaagatttctcatggtttggatttgggaaagtaaaatcaagacaaggtgttggcccagcctccgttatcagcaatgatgatgactctgccagcccactccatcacatctccaatgggagtaacactccatcttcttcggaaggtggcccagatgctgtcattattggaatgaccaagatccctgtcattgaaaatccccagtactttggcatcaccaacagtcagctcaagccagacacatttgttcagcacatcaagcgacataacattgttctgaaaagggagctaggcgaaggagcctttggaaaagtgttcctagctgaatgctataacctctgtcctgagcaggacaagatcttggtggcagtgaagaccctgaaggatgccagtgacaatgcacgcaaggacttccaccgtgaggccgagctcctgaccaacctccagcatgagcacatcgtcaagttctatggcgtctgcgtggagggcgaccccctcatcatggtctttgagtacatgaagcatggggacctcaacaagttcctcagggcacacggccctgatgccgtgctgatggctgagggcaacccgcccacggaactgacgcagtcgcagatgctgcatatagcccagcagatcgccgcgggcatggtctacctggcgtcccagcacttcgtgcaccgcgatttggccaccaggaactgcctggtcggggagaacttgctggtgaaaatcggggactttgggatgtcccgggacgtgtacagcactgactactacagggtcggtggccacacaatgctgcccattcgctggatgcctccagagagcatcatgtacaggaaattcacgacggaaagcgacgtctggagcctgggggtcgtgttgtgggagattttcacctatggcaaacagccctggtaccagctgtcaaacaatgaggtgatagagtgtatcactcagggccgagtcctgcagcgaccccgcacgtgcccccaggaggtgtatgagctgatgctggggtgctggcagcgagagccccacatgaggaagaacatcaagggcatccataccctccttcagaacttggccaaggcatctccggtctacctggacattctaggcgcggccgcggccatgagcaaagatggtaaaaagaagaaaaagaagtcaaagacaaagtgtgtaattatgtaa

SEQ ID NO: 6 - protein, 356 aa, Mus musculus
KCGQRSKFGINRPAVLAPEDGLAMSLHFMTLGGSSLSPTEGKGSGLQGHIMENPQYFSDTCVHHIKRQDIILKWELGEGAFGKVFLAECYNLLNDQDKMLVAVKALKEASENARQDFQREAELLTMLQHQHIVRFFGVCTEGGPLLMVFEYMRHGDLNRFLRSHGPDAKLLAGGEDVAPGPLGLGQLLAVASQVAAGMVYLASLHFVHRDLATRNCLVGQGLVVKIGDFGMSRDIYSTDYYRVGGRTMLPIRWMPPESILYRKFSTESDVWSFGVVLWEIFTYGKQPWYQLSNTEAIECITQGRELERPRACPPDVYAIMRGCWQREPQQRLSMKDVHARLQALAQAPPSYLDVLG

SEQ ID NO: 7 - DNA, 1068 bp, Mus musculus
aaatgtggacagaggagcaaatttgggatcaaccgccctgctgtattagctccagaggatgggctggccatgtccctacacttcatgacactgggtggcagttctctttcccctactgagggcaaaggctccggactccagggccacatcatggagaacccacagtacttcagtgatacctgtgtccatcacatcaagcgccaggacatcattctcaagtgggagctaggggagggagcctttggaaaggtctttctcgctgagtgctacaaccttctgaatgatcaggacaagatgctcgtggctgtcaaggcactgaaggaggcatccgagaatgctcggcaggactttcagcgtgaggccgagctgctcaccatgctacagcaccagcacatcgtgcgcttctttggagtctgcaccgagggtggcccgttgctcatggtctttgagtacatgcgccacggggacctcaaccgtttcctccggtcccacggacctgatgcaaagctgctggctggtggtgaggatgtggctcctggtcctctgggccttgggcagcttctggctgtggctagccaggtggctgctggtatggtatatctagccagcctgcactttgtgcaccgggatctggccacacgcaactgtctggtgggtcagggactagtggtgaagattggagactttggcatgagcagagacatctatagcacagactattaccgagtgggaggtcggaccatgctgcccatccgctggatgccacccgagagcatcctctaccgcaagttcagcaccgagagcgatgtgtggagctttggggtggtgctctgggagatcttcacctatggaaagcagccctggtaccagctctccaacactgaggcgatcgagtgtatcacgcagggccgggagctggagcggccgcgcgcctgccctcctgatgtctacgccatcatgcgaggctgctggcagcgagaaccgcagcaacgcctcagcatgaaggatgtgcacgcgcgtctgcaggccctggcacaggcgccacccagttacctggacgttctgggc

SEQ ID NO: 8 - DNA, 1182 bp, Artificial Sequence
  nucleic acid sequence coding for CA-TrekA
atggcatcaatgcagaagctgatctcagaggaggacctggtcgacaaatgtggacagaggagcaaatttgggatcaaccgccctgctgtattagctccagaggatgggctggccatgtccctacacttcatgacactgggtggcagttctctttcccctactgagggcaaaggctccggactccagggccacatcatggagaacccacagtacttcagtgatacctgtgtccatcacatcaagcgccaggacatcattctcaagtgggagctaggggagggagcctttggaaaggtctttctcgctgagtgctacaaccttctgaatgatcaggacaagatgctcgtggctgtcaaggcactgaaggaggcatccgagaatgctcggcaggactttcagcgtgaggccgagctgctcaccatgctacagcaccagcacatcgtgcgcttctttggagtctgcaccgagggtggcccgttgctcatggtctttgagtacatgcgccacggggacctcaaccgtttcctccggtcccacggacctgatgcaaagctgctggctggtggtgaggatgtggctcctggtcctctgggccttgggcagcttctggctgtggctagccaggtggctgctggtatggtatatctagccagcctgcactttgtgcaccgggatctggccacacgcaactgtctggtgggtcagggactagtggtgaagattggagactttggcatgagcagagacatctatagcacagactattaccgagtgggaggtcggaccatgctgcccatccgctggatgccacccgagagcatcctctaccgcaagttcagcaccgagagcgatgtgtggagctttggggtggtgctctgggagatcttcacctatggaaagcagccctggtaccagctctccaacactgaggcgatcgagtgtatcacgcagggccgggagctggagcggccgcgcgcctgccctcctgatgtctacgccatcatgcgaggctgctggcagcgagaaccgcagcaacgcctcagcatgaaggatgtgcacgcgcgtctgcaggccctggcacaggcgccacccagttacctggacgttctgggctctagagccatgagcaaagatggtaaaaagaagaaaaagaagtcaaagacaaagtgtgtaattatgtaa

SEQ ID NO: 9 - protein, 371 aa, Mus musculus
KYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILG

SEQ ID NO: 10 - DNA, 1113 bp, Mus musculus
aagtatggtcgccggtccaaatttggaatgaagggtcctgtggctgttatcagtggagaggaggactcagccagcccactgcatcacatcaaccatggcatcactacaccatcatcgttggatgctgggcctgacacagtggtcattggcatgacccgcatcccagtcattgagaacccccagtacttccgtcagggtcacaattgccacaagccagacacatatgttcagcacatcaagaggagagacatcgtgttgaagagagaattgggtgagggagcctttgggaaggtcttcctggctgagtgctacaatctaagccccaccaaagacaagatgctagtggcagtgaaggccctgaaggatcccaccttggctgccaggaaggatttccagagggaggctgagctgctcacgaacctgcagcatgagcatattgtcaagttctatggggtgtgtggtgatggtgacccactcatcatggtctttgaatacatgaagcatggagaccttaacaagttcctcagggcccatgggccagatgccatgatcctcgtggatggacagccacgtcaggccaagggggagctagggctctctcagatgctccacatcgccagtcagatagcctcgggcatggtgtacctggcttcccagcactttgtacaccgggacctggccacgaggaactgcctggttggagccaatctactagtgaagattggagattttggcatgtccagggacgtctacagtactgattactacagggtgggaggacacaccatgctccccatccgctggatgccccctgaaagcataatgtaccggaagttcaccacagagagtgatgtctggagcttcggggttattctttgggagatctttacctatgggaagcaaccatggttccagctttccaacacggaggtcattgaatgcatcacccaaggccgtgtcttggagagacccagagtctgccctaaagaagtgtatgatgtcatgctggggtgctggcagagggaaccacagcagcggctgaatattaaggagatctacaaaatcctccatgctttggggaaggccaccccgatctacctggacattcttggc

SEQ ID NO: 11 - DNA, 1227 bp, Artificial Sequence
  nucleic acid sequence coding for CA-TrkC
atggcatcaatgcagaagctgatctcagaggaggacctggtcgacaagtatggtcgccggtccaaatttggaatgaagggtcctgtggctgttatcagtggagaggaggactcagccagcccactgcatcacatcaaccatggcatcactacaccatcatcgttggatgctgggcctgacacagtggtcattggcatgacccgcatcccagtcattgagaacccccagtacttccgtcagggtcacaattgccacaagccagacacatatgttcagcacatcaagaggagagacatcgtgttgaagagagaattgggtgagggagcctttgggaaggtcttcctggctgagtgctacaatctaagccccaccaaagacaagatgctagtggcagtgaaggccctgaaggatcccaccttggctgccaggaaggatttccagagggaggctgagctgctcacgaacctgcagcatgagcatattgtcaagttctatggggtgtgtggtgatggtgacccactcatcatggtctttgaatacatgaagcatggagaccttaacaagttcctcagggcccatgggccagatgccatgatcctcgtggatggacagccacgtcaggccaagggggagctagggctctctcagatgctccacatcgccagtcagatagcctcgggcatggtgtacctggcttcccagcactttgtacaccgggacctggccacgaggaactgcctggttggagccaatctactagtgaagattggagattttggcatgtccagggacgtctacagtactgattactacagggtgggaggacacaccatgctccccatccgctggatgccccctgaaagcataatgtaccggaagttcaccacagagagtgatgtctggagcttcggggttattctttgggagatctttacctatgggaagcaaccatggttccagctttccaacacggaggtcattgaatgcatcacccaaggccgtgtcttggagagacccagagtctgccctaaagaagtgtatgatgtcatgctggggtgctggcagagggaaccacagcagcggctgaatattaaggagatctacaaaatcctccatgctttggggaaggccaccccgatctacctggacattcttggctctagagccatgagcaaagatggtaaaaagaagaaaaagaagtcaaagacaaagtgtgtaattatgtaa

SEQ ID NO: 12 - protein, 357 aa, Homo sapiens
KCGRRNKFGINRPAVLAPEDGLAMSLHFMTLGGSSLSPTEGKGSGLQGHIIENPQYFSDACVHHIKRRDIVLKWELGEGAFGKVFLAECHNLLPEQDKMLVAVKALKEASESARQDFQREAELLTMLQHQHIVRFFGVCTEGRPLLMVFEYMRHGDLNRFLRSHGPDAKLLAGGEDVAPGPLGLGQLLAVASQVAAGMVYLAGLHFVHRDLATRNCLVGQGLVVKIGDFGMSRDIYSTDYYRVGGRTMLPIRWMPPESILYRKFTTESDVWSFGVVLWEIFTYGKQPWYQLSNTEAIDCITQGRELERPRACPPEVYAIMRGCWQREPQQRHSIKDVHARLQALAQAPPVYLDVLGZ

SEQ ID NO: 13 - DNA, 1071 bp, Homo sapiens
aaatgtggacggagaaacaagtttgggatcaaccgcccggctgtgctggctccagaggatgggctggccatgtccctgcatttcatgacattgggtggcagctccctgtcccccaccgagggcaaaggctctgggctccaaggccacatcatcgagaacccacaatacttcagtgatgcctgtgttcaccacatcaagcgccgggacatcgtgctcaagtgggagctgggggagggcgcctttgggaaggtcttccttgctgagtgccacaacctcctgcctgagcaggacaagatgctggtggctgtcaaggcactgaaggaggcgtccgagagtgctcggcaggacttccagcgtgaggctgagctgctcaccatgctgcagcaccagcacatcgtgcgcttcttcggcgtctgcaccgagggccgccccctgctcatggtctttgagtatatgcggcacggggacctcaaccgcttcctccgatcccatggacctgatgccaagctgctggctggtggggaggatgtggctccaggccccctgggtctggggcagctgctggccgtggctagccaggtcgctgcggggatggtgtacctggcgggtctgcattttgtgcaccgggacctggccacacgcaactgtctagtgggccagggactggtggtcaagattggtgattttggcatgagcagggatatctacagcaccgactattaccgtgtgggaggccgcaccatgctgcccattcgctggatgccgcccgagagcatcctgtaccgtaagttcaccaccgagagcgacgtgtggagcttcggcgtggtgctctgggagatcttcacctacggcaagcagccctggtaccagctctccaacacggaggcaatcgactgcatcacgcagggacgtgagttggagcggccacgtgcctgcccaccagaggtctacgccatcatgcggggctgctggcagcgggagccccagcaacgccacagcatcaaggatgtgcacgcccggctgcaagccctggcccaggcacctcctgtctacctggatgtcctgggctag

SEQ ID NO: 14 - protein, 372 aa, Homo sapiens
KYGRRSKFGMKGPVAVISGEEDSASPLHHINHGITTPSSLDAGPDTVVIGMTRIPVIENPQYFRQGHNCHKPDTYVQHIKRRDIVLKRELGEGAFGKVFLAECYNLSPTKDKMLVAVKALKDPTLAARKDFQREAELLTNLQHEHIVKFYGVCGDGDPLIMVFEYMKHGDLNKFLRAHGPDAMILVDGQPRQAKGELGLSQMLHIASQIASGMVYLASQHFVHRDLATRNCLVGANLLVKIGDFGMSRDVYSTDYYRVGGHTMLPIRWMPPESIMYRKFTTESDVWSFGVILWEIFTYGKQPWFQLSNTEVIECITQGRVLERPRVCPKEVYDVMLGCWQREPQQRLNIKEIYKILHALGKATPIYLDILGZ

SEQ ID NO: 15 - DNA, 1116 bp, Homo sapiens
aaatatggtcgacggtccaaatttggaatgaagggtcccgtggctgtcatcagtggtgaggaggactcagccagcccactgcaccacatcaaccacggcatcaccacgccctcgtcactggatgccgggcccgacactgtggtcattggcatgactcgcatccctgtcattgagaacccccagtacttccgtcagggacacaactgccacaagccggacacgtatgtgcagcacattaagaggagagacatcgtgctgaagcgagaactgggtgagggagcctttggaaaggtcttcctggccgagtgctacaacctcagcccgaccaaggacaagatgcttgtggctgtgaaggccctgaaggatcccaccctggctgcccggaaggatttccagagggaggccgagctgctcaccaacctgcagcatgagcacattgtcaagttctatggagtgtgcggcgatggggaccccctcatcatggtctttgaatacatgaagcatggagacctgaataagttcctcagggcccatgggccagatgcaatgatccttgtggatggacagccacgccaggccaagggtgagctggggctctcccaaatgctccacattgccagtcagatcgcctcgggtatggtgtacctggcctcccagcactttgtgcaccgagacctggccaccaggaactgcctggttggagcgaatctgctagtgaagattggggacttcggcatgtccagagatgtctacagcacggattattacagggtgggaggacacaccatgctccccattcgctggatgcctcctgaaagcatcatgtaccggaagttcactacagagagtgatgtatggagcttcggggtgatcctctgggagatcttcacctatggaaagcagccatggttccaactctcaaacacggaggtcattgagtgcattacccaaggtcgtgttttggagcggccccgagtctgccccaaagaggtgtacgatgtcatgctggggtgctggcagagggaaccacagcagcggttgaacatcaaggagatctacaaaatcctccatgctttggggaaggccaccccaatctacctggacattcttggctag

(19) United States
(12) Patent Application Publication
(10) Pub. No.: US 2022/0162572 A1
(43) Pub. Date: May 26, 2022

(54) NUCLEIC ACID CONSTRUCT ENCODING Trk FRAGMENT AND USE THEREOF

(71) Applicant: TOKYO METROPOLITAN INSTITUTE OF MEDICAL SCIENCE, Tokyo (JP)

(72) Inventors: Kazuhiko NAMEKATA, Tokyo (JP); Takayuki HARADA, Tokyo (JP)

(21) Appl. No.: 17/436,256

(22) PCT Filed: Mar. 4, 2020
(86) PCT No.: PCT/JP2020/009259

§ 371 Date: Sep. 3, 2021

(30) Foreign Application Priority Data
Mar. 4, 2019 (JP) 2019-038503

(51) Int. Cl. C12N 9/12; C12N 15/62; C12N 15/86; C07K 14/705; A61K 38/45; A61P 25/00; A61P 27/02; A61P 27/06

(52) U.S. Cl. CPC C12N 9/12; C12N 15/625; C12Y 207/10001; C12N 15/86; C07K 14/705; A61K 38/00; A61P 25/00; A61P 27/02; A61P 27/06; C12N 2750/14143; C07K 2319/035; A61K 38/45

(57) ABSTRACT

The present invention provides: a novel nucleic acid construct encoding a functional fragment of a Trk family member; and use of the nucleic acid construct. A nucleic acid construct in accordance with an embodiment of the present invention encodes a fusion polypeptide including an intracellular domain of Trk and a membrane localization sequence.

### TECHNICAL FIELD

The present invention relates to a nucleic acid construct encoding a functional fragment of a Trk family member and to use of the nucleic acid construct.

### BACKGROUND ART

Nerve growth factor (NGF), brain-derived neurotrophic factor (BDNF), neurotrophin-3 (NT-3), NT-4/5, and the like are known as neurotrophic factors which play important roles in neural cell survival, axonal outgrowth, generation of a central nervous system, and the like (Non-patent Literature 1).

TrkB is known as a high-affinity receptor for the above BDNF. Typically, an extracellular domain of TrkB forms a dimer so that TrkB can receive BDNF. However, there is also a truncated form of TrkB that lacks a tyrosine kinase domain in a cell, and the function of the truncated form of TrkB is not fully understood (Non-patent Literature 2).

The inventors of the present invention have studied neurotrophic factors for years and have reported that control of signaling between glia cells and signaling between a glia cell and a neuron is important for neuroprotection (Non-patent Literatures 3 to 5).

Further, as the inventors continued the study of neuroprotection and axon regeneration by TrkB signaling, the inventors have been focusing on the functions and the like of Dock3, which is a guanine nucleotide exchange factor (GEF) activated by TrkB signaling (Non-patent Literatures 6 to 9).

### CITATION LIST

### Patent Literature

[Patent Literature 1]
International Publication WO2018/185468 (International Publication Date: Oct. 11, 2018)

### Non-Patent Literature

[Non-Patent Literature 1]
Park H, Poo M M. Neurotrophin regulation of neural circuit development and function. Nat Rev Neurosci 14(1): 7-23, 2013.

### Non-Patent Literature 2

Fenner B M. Truncated TrkB: beyond a dominant negative receptor. Cytokine Growth Factor Rev 23(1-2): 15-24, 2012.

[Non-Patent Literature 3]
Harada T, Harada C, Nakayama N, Okuyama S, Yoshida K, Kohsaka S, Matsuda H, Wada K. Modification of glial-neuronal cell interactions prevents photoreceptor apoptosis during light-induced retinal degeneration. Neuron 26(2): 533-541, 2000.

[Non-Patent Literature 4]
Harada T, Harada C, Kohsaka S, Wada E, Yoshida K, Ohno S, Mamada H, Tanaka K, Parada L F, Wada K.

Microglia-Muller glia cell interactions control neurotrophic factor production during light-induced retinal degeneration. J Neurosci 22(21): 9228-9236, 2002.

[Non-Patent Literature 5]
Harada C, Guo X, Namekata K, Kimura A, Nakamura K, Tanaka K, Parada L F, Harada T. Glia- and neuronspecific functions of TrkB signalling during retinal degeneration and regeneration. Nat Commun 2: 189, 2011.

[Non-Patent Literature 6]
Namekata K, Harada C, Taya C, Guo X, Kimura H, Parada L F, Harada T. Dock3 induces axonal outgrowth by stimulating membrane recruitment of the WAVE complex. Proc Natl Acad Sci USA 107(16): 7586-7591, 2010.

[Non-Patent Literature 7]
Namekata K, Harada C, Guo X, Kimura A, Kittaka D, Watanabe H, Harada T. Dock3 stimulates axonal outgrowth via GSK-3β-mediated microtubule assembly. J Neurosci 32(1): 264-274, 2012.

[Non-Patent Literature 8]
Namekata K, Kimura A, Kawamura K, Harada C, Harada T. Dock GEFs and their therapeutic potential: neuroprotection and axon regeneration. Prog Retin Eye Res 43: 1-16, 2014.

[Non-Patent Literature 9]
Kazuhiko NAMEKATA, Takayuki HARADA. Shinkei Jikusaku no Saisei ni okeru Dock3 no Kino (Function of Dock3 in Regeneration of Neural Axons). SEIKAGAKU (Biochemistry) 84(5), 368-373, 2012.

### SUMMARY OF INVENTION

### Technical Problem

As described above, the important roles of TrkB in neuroprotection and the like are beginning to be understood from various viewpoints.

However, it is difficult to induce high-level expression of TrkB with use of a genetic vector having a full-length TrkB gene incorporated therein. This is because the full-length TrkB gene has a relatively large size, so that a workable size of a promoter and the like are restricted.

Further, in treatment of a nerve-related disease, it is considered necessary to not only cause overexpression of TrkB but also periodically administer BDNF at the same time. As such, no study of gene therapy utilizing TrkB has been put into practice.

A recent report shows an expression vector that has incorporated therein a gene encoding a fusion protein of BDNF and TrkB (Patent Literature 1). However, considering the molecular size of the fusion protein, it is presumed that the expression level is not very high.

The present invention has been accomplished in light of the above problems. An objective of the present invention is to provide a novel nucleic acid construct encoding a functional fragment of a Trk family member, and use of the nucleic acid construct.

### Solution to Problem

In order to attain the objective, the present invention encompasses the following aspects.

(i) A nucleic acid construct encoding a fusion polypeptide, the fusion polypeptide including: an intracellular domain of Trk; and a membrane localization sequence.

(ii) A method for cell activation, including the step of causing an intracellular domain of Trk to be localized at a cell membrane via a membrane localization sequence in a cell.

(iii) A fusion polypeptide including: an intracellular domain of Trk; and a membrane localization sequence.

### Advantageous Effects of Invention

According to an aspect of the present invention, it is possible to provide a novel nucleic acid construct encoding a functional fragment of a Trk family member, use of the nucleic acid construct, and the like.

### BRIEF DESCRIPTION OF DRAWINGS

FIG. 1 is a view illustrating a structure of constitutive active TrkB (CA-TrkB).

FIG. 2 is a view illustrating an adeno-associated virus vector prepared in Example 1.

FIG. 3 is a view illustrating results (an expression level of CA-TrkB) of Example 1.

FIG. 4 is a view illustrating other results (intracellular signaling by CA-TrkB) of Example 1.

FIG. 5 is a view illustrating results (protection of retinal ganglion cells by CA-TrkB) of Example 2.

FIG. 6 is a view illustrating results (optic nerve axon regeneration by CA-TrkB) of Example 2.

FIG. 7 is a view illustrating results (protection of retinal ganglion cells by CA-TrkB with use of glaucoma model mice) of Example 3.

FIG. 8 is a view illustrating results (examination of signaling with use of mutants of CA-TrkB) of Example 4.

FIG. 9 is a view relating to Example 5 and illustrating structures of constitutive active TrkA and TrkC and results (intracellular signaling by CA-TrkA and CA-TrkC) of Example 5.

FIG. 10 is a view illustrating a base sequence of a gene fragment encoding iSH-TrkB in an Example.

FIG. 11 is a view illustrating a base sequence of a gene fragment encoding CA-TrkA in an Example.

FIG. 12 is a view illustrating a base sequence of a gene fragment encoding CA-TrkC in an Example.

FIG. 13 is a view illustrating results (measurement of a visual evoked potential) of Example 6.

### DESCRIPTION OF EMBODIMENTS

### Definitions of Terms and the Like

As used herein, the term “polynucleotide” can be interpreted as meaning “nucleic acid” or “nucleic acid molecule”. The term “polynucleotide”, unless otherwise specified, includes in its scope a polynucleotide that contains a known analog of a naturally-occurring nucleotide capable of functioning similarly to the naturally-occurring nucleotide. The term “base sequence” can be interpreted as meaning “nucleic acid sequence” or “nucleotide sequence”. Unless otherwise indicated, the term “base sequence” is intended to mean a deoxyribonucleotide sequence or a ribonucleotide sequence. The polynucleotide may have a single-stranded structure or a double-stranded structure, and may be a sense strand or an antisense strand in the case of a single strand.

As used herein, the term “gene” refers to a “polynucleotide” encoding a protein.

As used herein, the term “expression regulatory region” of a gene refers to a “polynucleotide” that regulates expression of the gene. Examples of the “expression regulatory region” include a promoter region and an enhancer region.

As used herein, the term “expression cassette” refers to an expression unit including (i) an expression regulatory region that is functional in an expression host and (ii) a polynucleotide that is operably linked to the expression regulatory region. In the expression cassette, the polynucleotide is preferably a gene or a gene fragment. An example of the expression cassette is an expression unit in which the above expression regulatory region is linked to the above polynucleotide in a genetically engineered manner. The term “operably linked” refers to a state in which expression of a polynucleotide is regulated with use of an expression regulatory sequence. The expression cassette may be in the form of an expression vector.

As used herein, the term “polypeptide” is interchangeable with “protein”. A “polypeptide” includes a structure of amino acids linked by a peptide bond. A “polypeptide” may further include a structure of a sugar chain or an isoprenoid group and the like. The term “polypeptide”, unless otherwise specified, includes in its scope a polypeptide that contains a known analog of a naturally-occurring amino acid having a function as well as the naturally-occurring amino acid.

[1. Nucleic Acid Construct]

A nucleic acid construct in accordance with an embodiment of the present invention is a nucleic acid construct encoding a fusion polypeptide including an intracellular domain of Trk and a membrane localization sequence. The present invention has been made based on the surprising knowledge that causing the nucleic acid construct to be expressed in a cell enables activation of an intracellular signaling pathway downstream of Trk without administration of a neurotrophic factor (neurotrophin). That is, the present invention relates to a novel cell activation technique of causing the intracellular domain of Trk to be localized at a cell membrane via the membrane localization sequence of the fusion polypeptide encoded by the nucleic acid construct.

The nucleic acid construct in accordance with the present embodiment, for example, provides effects typified by the following.

1) There is no need for the overexpression or administration of a corresponding neurotrophic factor.
2) The molecular size required for activation can be considerably reduced in comparison with full-length Trk. This enables high-level expression of molecules. Further, since the degree of freedom in designing a nucleic acid construct is considerably increased, it is possible, for example, to more easily achieve high-level expression, improvement in gene introduction efficiency, and the like.

(Fusion Polypeptide)

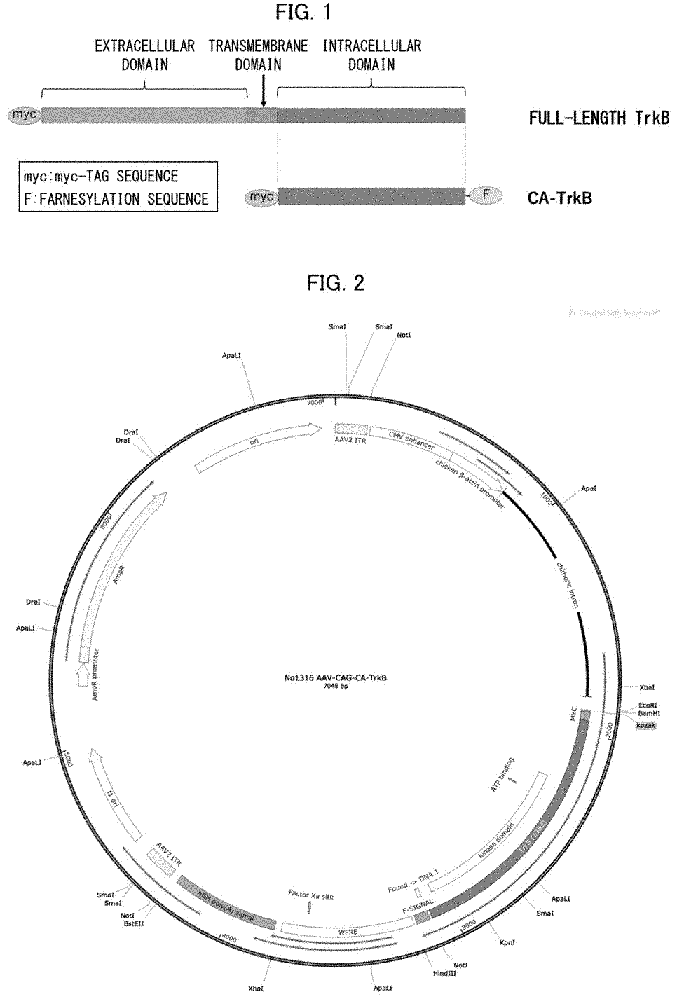

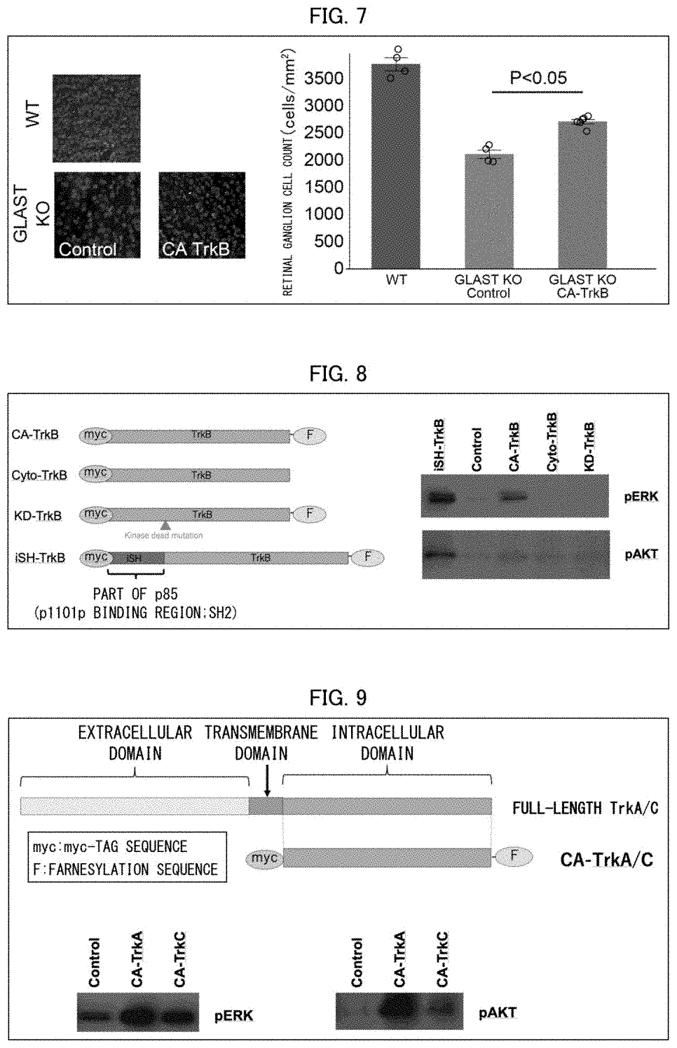

The fusion polypeptide encoded by the nucleic acid construct in accordance with the present embodiment includes (1) an intracellular domain of Trk and (2) a membrane localization sequence, each of which will be described later. It may be preferable that the membrane localization sequence be provided on the N-terminus side of the fusion polypeptide or on the C-terminus side of the fusion polypeptide, depending on the characteristics of the membrane localization sequence. In relation to the intracellular domain of Trk, the membrane localization sequence may be provided on the N-terminus side of the intracellular domain (on a side close to a transmembrane domain) or may be provided on the C-terminus side of the intracellular domain. Note that in constitutive active Trks shown in Examples, a membrane localization sequence is provided on a side that is the C-terminus side of a fusion polypeptide and is the C-terminus side of an intracellular domain of Trk. Thus, in each of the constitutive active Trks shown in the Examples, the intracellular domain of Trk is located on a side opposite to the side on which an intracellular domain is typically located, with respect to a cell membrane (see also FIGS. 1 and 9).

In one example, the fusion polypeptide may include “another sequence” other than “the intracellular domain of Trk and the membrane localization sequence”. Examples of the “another sequence” include a spacer sequence and an SH2 sequence of p85. The position in which the “another sequence” is inserted can be set according to the purpose of inserting the “another sequence”. For example, the position can be between the intracellular domain of Trk and the membrane localization sequence, or on the N-terminus side of the fusion polypeptide (in this case, it may be preferable that the membrane localization sequence be provided on the C-terminus side), or on the C-terminus side of the fusion polypeptide (in this case, it may be preferable that the membrane localization sequence be provided on the N-terminus side).

In a preferable example, the number of amino acids in the fusion polypeptide is in a range of 300 to 650, and may be more preferably in a range of 330 to 630. In a preferable example, the number of amino acids in “the intracellular domain of Trk and the membrane localization sequence” in the fusion polypeptide is in a range of 300 to 450, and may be more preferably in a range of 330 to 440, 350 to 440, 360 (or 370) to 440, or 360 (or 370) to 410. The fusion polypeptide having a small size has advantages such as, for example, (1) an increased degree of freedom in designing other features in the nucleic acid construct (that is, features other than a region encoding the fusion polypeptide) and (2) an improved expression efficiency of the fusion polypeptide. In particular, having a wider range of options for a promoter and the like in the nucleic acid construct significantly contributes to improving the expression efficiency of the fusion polypeptide.

In one example, the fusion polypeptide may include a part of an extracellular domain of Trk and/or a part of a transmembrane domain. In a preferable example, the fusion polypeptide does not include the extracellular domain of Trk and/or the transmembrane domain. In a more preferable example, the fusion polypeptide includes neither the extracellular domain of Trk nor the transmembrane domain.

(Intracellular Domain of Trk)

In the present embodiment, the intracellular domain of Trk refers to (i) a polypeptide corresponding to an intracellular domain of a high-affinity receptor (Trk) for a neurotrophic factor, which high-affinity receptor is a single-span transmembrane protein, and (ii) a polypeptide functionally equivalent to this polypeptide. The intracellular domain is also referred to as a cytoplasmic domain.

Currently, the following three types of Trk family members have been reported: TrkA, TrkB, and TrkC. The extracellular domains of TrkA, TrkB, and TrkC are each a domain receiving a neurotrophic factor, and the intracellular domains of TrkA, TrkB, and TrkC each have tyrosine kinase activity. In a neural cell, TrkA, TrkB, and TrkC respectively receive corresponding neurotrophic factors. The reception of the neurotrophic factors triggers activation of respective intracellular signaling pathways downstream of TrkA, TrkB, and TrkC. Note that TrkA is a receptor for NGF, TrkB is a receptor for NT-4/5, and TrkC is a receptor for NT-3.

The intracellular domain of Trk is only required to (1) have tyrosine kinase activity and/or (2) be capable of activating an intracellular signaling pathway downstream of Trk in a case where the intracellular domain is localized at a cell membrane. The activation of the intracellular signaling pathway downstream of Trk can be recognized by a known technique, for example, by a technique of recognizing the activation in the form of an increased expression of at least one selected from Ras, ERK1/2, PI3K, Akt, phospholipase C-γ, and the like.

Examples of the intracellular domain of Trk preferably include, but are not particularly limited to, the following.

1) A polypeptide having an amino acid sequence represented by any one of SEQ ID NO. 2, 6, 9, 12, or 14. Note that SEQ ID NO. 2 is an example of an amino acid sequence of an intracellular domain of human-derived TrkB, SEQ ID NO. 6 is an example of an amino acid sequence of an intracellular domain of mouse-derived TrkA, SEQ ID NO. 9 is an example of an amino acid sequence of an intracellular domain of mouse-derived TrkC, SEQ ID NO. 12 is an example of an amino acid sequence of an intracellular domain of human-derived TrkA, and SEQ ID NO. 14 is an example of an amino acid sequence of an intracellular domain of human-derived TrkC.
2) A polypeptide functionally equivalent to a polypeptide having an amino acid sequence represented by any one of SEQ ID NO. 2, 6, 9, 12, or 14. Examples of the functionally equivalent polypeptide include a polypeptide having an amino acid sequence having a sequence identity of 90% or more with respect to an amino acid sequence represented by any one of SEQ ID NO: 2, 6, or 9. The sequence identity is more preferably 91% or more, 92% or more, 93% or more, 94% or more, 95% or more, 96% or more, 97% or more, 98% or more, or 99% or more.
A 104th amino acid (Lys) in the amino acid sequence represented by SEQ ID NO: 6 is an amino acid sequence essential for tyrosine kinase activity or for activation of an intracellular signaling pathway downstream of TrkA, and thus preferably includes no mutation.
Similarly, a 134th amino acid (Lys) in the amino acid sequence represented by SEQ ID NO: 2 is an amino acid sequence essential for tyrosine kinase activity or for activation of an intracellular signaling pathway downstream of TrkB, and thus preferably includes no mutation.
Similarly, a 118th amino acid (Lys) in the amino acid sequence represented by SEQ ID NO: 9 is an amino acid sequence essential for tyrosine kinase activity or for activation of an intracellular signaling pathway downstream of TrkC, and thus preferably includes no mutation.
Similarly, a 104 amino acid (Lys) in the amino acid sequence represented by SEQ ID NO: 12 is an amino acid sequence essential for tyrosine kinase activity or for activation of an intracellular signaling pathway downstream of TrkA, and thus preferably includes no mutation.
Similarly, a 118th amino acid (Lys) in the amino acid sequence represented by SEQ ID NO: 14 is an amino acid sequence essential for tyrosine kinase activity or for activation of an intracellular signaling pathway downstream of TrkC, and thus preferably includes no mutation.

(Membrane Localization Sequence)

The membrane localization sequence is a peptide sequence for causing the above fusion polypeptide to be localized at a cell membrane. The membrane localization sequence preferably induces lipid modification of an amino acid. The type of the lipid modification of an amino acid is not particularly limited, but can be, for example, (1) fatty-acylation, (2) prenylation, (3) glycosylphosphatidylinositolation, (4) cholesterolation, or the like, and more specifically myristoylation, palmitoylation, farnesylation, geranylgeranylation, or the like, and preferably farnesylation or geranylgeranylation.

Specific examples of an amino acid sequence motif constituting the membrane localization sequence include the following:

(1) Met-Gly-X-X-X-Ser-X-X-X (an N-terminus side lipid modification motif (N-myristoylation)); and (2) a CAAX motif, a CC motif, a CXC motif, a CCXX motif (a C-terminus side lipid modification motif) where X is any amino acid, A is an aliphatic amino acid (e.g., isoleucine, leucine, valine, alanine, methionine), and C is cysteine.

(Aspect of Nucleic Acid Construct)

An aspect of the nucleic acid construct in accordance with the present embodiment is a gene encoding the above fusion polypeptide including the intracellular domain of Trk and the membrane localization sequence. An example of the gene can be a gene having a base sequence represented by any one of SEQ ID NO: 4, 5, 8, or 11, or can be a gene having a base sequence having a sequence identity of 85% or more, 90% or more, 91% or more, 92% or more, 93% or more, 94% or more, 95% or more, 96% or more, 97% or more, 98% or more, or 99% or more with respect to a base sequence represented by any one of SEQ ID NO: 4, 5, 8, or 11. Note that an example of a gene encoding the intracellular domain of Trk included in the above fusion polypeptide can be a gene having a base sequence represented by any one of SEQ ID NO: 3, 7, 10, 13, or 15, or can be a gene having a base sequence having a sequence identity of 85% or more, 90% or more, 91% or more, 92% or more, 93% or more, 94% or more, 95% or more, 96% or more, 97% or more, 98% or more, or 99% or more with respect to a base sequence represented by any one of SEQ ID NO: 3, 7, 10, 13, or 15.

Another aspect of the nucleic acid construct in accordance with the present embodiment is an expression cassette which causes the above gene to be expressed. The expression cassette is preferably an expression vector, and more preferably a virus vector such as a retrovirus vector, a lentivirus vector, an adenovirus vector, an adeno-associated virus vector, a herpesvirus vector, and a vaccinia virus vector. Among these examples, from the viewpoint of use in a gene therapy against a nerve-related disease, the adeno-associated virus vector is particularly preferable. In particular, from the viewpoint of use in a gene therapy against a nerve-related disease, it may be preferable that the gene be an adeno-associated virus vector such as a serotype 1 (AAV1) having a tissue tropism for a central nervous system and the like, a serotype 2 (AAV2) having a broad tissue tropism, and a serotype 5 (AAV5) having a tissue tropism for a central nervous system, retina, and the like.

The above expression cassette includes any expression regulatory region that is functional in an expression host. A promoter serving as the expression regulatory region is not limited to a particular type, but specifically, for example, it may be preferable that the promoter be a promoter that enables high-level expression in a mammal (in particular, a human) or selectively functions in a specific tissue, such as a CAG promoter, a CMV promoter, and SYN (synapsin) I. Among these examples, the CAG promoter may be particularly preferable as the promoter. In the expression cassette, the promoter is provided on an upstream side of the gene.

The above expression cassette may further include, as necessary, a functional sequence that has an effect of, for example, increasing the stability of mRNA produced from transcription and serves to achieve an improvement of gene expression and the like. Examples of the functional sequence include a WPRE (woodchuck hepatitis virus posttranscriptional regulatory element) sequence. In the expression cassette, the WPRE sequence is provided on a downstream side of the gene.

Other examples of the functional sequence that can be included in the above expression cassette include a poly-A tail addition signal sequence. Examples of the poly-A tail addition signal sequence include a poly-A tail addition signal sequence of simian virus 40 and a poly-A tail addition signal sequence of human growth hormone. The poly-A tail addition signal sequence of human growth hormone may be more preferable. In the expression cassette, the WPRE sequence is provided on a downstream side of the gene.

The above expression cassette may further include inverted terminal repeat sequences (ITRs) and the like. ITRs are provided in the expression cassette so as to sandwich an entire transcription unit (from the promoter to the poly-A tail addition signal sequence) therebetween.

[2. Pharmaceutical Composition]

A pharmaceutical composition in accordance with an embodiment of the present invention is a gene therapeutic drug containing the above nucleic acid construct. In one example, the pharmaceutical composition is a drug for a nerve-related disease. In a more specific example, the pharmaceutical composition is for neuroprotection or neuroregeneration. The pharmaceutical composition exhibits a high neuroprotection effect and a high axon regeneration effect and can be used for treatment and/or prevention of a nerve-related disease. The scope of the present invention encompasses a method for treatment and/or prevention of a nerve-related disease, including administration of an effective amount of the pharmaceutical composition to an administration target.

(Administration Target)

A human or a non-human animal to be an administration target is preferably any one selected from the group consisting of mammals including a human. The type of a mammal to be an administration target is not particularly limited, but can be a laboratory animal such as a mouse, a rat, a rabbit, a guinea pig, and a primate other than a human; a pet animal such as a dog and a cat; a farm animal such as a cow and a horse; or a human, and particularly preferably a human. The human or the non-human animal to be an administration target has, for example, a nerve-related disease described later.

(Nerve-Related Disease)

The nerve-related disease to be treated and/or prevented is not limited to a particular type, but refers to a pathological condition in which the function of a neural cell is impaired due to denaturation of the neural cell and/or cell death of the neural cell. The causes of the denaturation and cell death of the neural cell are not particularly limited and include a damage of the nervous system resulting from an accident or the like.

Examples of a nerve-related disease of an optic nerve system include: a neurodegenerative disease such as glaucoma, retinitis pigmentosa, age-related macular degeneration, and diabetic retinopathy; and a damage of an optic nerve system resulting from an accident or the like. In particular, a disease accompanied by degeneration of retinal ganglion cells typified by glaucoma and a damage of an optic nerve system (optic nerve injury) may be preferable as a disease to which the present invention is applied. Examples of a nerve-related disease of an auditory nervous system include: a sensory nerve degenerative disease such as nervous deafness; and a damage of an auditory nervous system resulting from an accident or the like.

(Administration Method, Dosage, Number of Times of Administration, and the Like)

A method of administering the pharmaceutical composition is not particularly limited. The pharmaceutical composition may be administered topically by a method such as injection (with use of a syringe, an injection pump, or the like), instillation to an eye, transdermal administration, or sublingual administration, or may be administered systemically by a method such as oral administration, intravascular administration such as intravenous administration or intraarterial administration, or enteral administration. In a preferable aspect of administration, the pharmaceutical composition is administered topically to a part in the vicinity of a nervous system to be subjected to treatment or the like.

The dosage (effective amount) of the pharmaceutical composition may be set as appropriate according to the age and sex of the human or animal to be an administration target, the symptom, the administration route, the number of times of administration, and the like. As necessary, an in vivo assay with use of the pharmaceutical composition may be carried out in advance to determine the dosage without undue experiment.

The number of times of administration of the pharmaceutical composition is not particularly limited as long as an effect is obtained, and can be set as appropriate, for example, according to the dosage, the administration route, the symptom, the age and sex of the human or animal.

(Component Other than Nucleic Acid Construct)

A pharmaceutical composition in accordance with an embodiment of the present invention can contain at least the above nucleic acid construct and a pharmaceutically-acceptable carrier. The pharmaceutically-acceptable carrier is not particularly limited, but is preferably one that does not substantially inhibit the function of the nucleic acid construct and does not have a substantial adverse effect on the human or non-human animal to be an administration target. It may be preferable that the pharmaceutical composition contain a liquid carrier such as water. In one example, the pharmaceutical composition may be a liposomal preparation.

Examples of the component constituting the above pharmaceutical composition further include, but are not particularly limited to, a lubricant, a preservative, a stabilizer, a wetting agent, an emulsifier, an osmotic pressure controlling salt, a buffer agent, a coloring agent, an antioxidant, and a viscosity modifier.

[3. Cell Activation Method and the Like]

A method for cell activation in accordance with an embodiment of the present invention is a method including the step (localization step) of causing an intracellular domain of Trk to be localized at a cell membrane via a membrane localization sequence in a cell. The localization step can typically be carried out by causing one selected from a nucleic acid construct described in the section [1. Nucleic acid construct] and a pharmaceutical composition described in the section [2. Pharmaceutical composition] to be expressed in a neural cell. The type of the neural cell is not particularly limited, and examples of the neural cell include a cell of a central nervous system, a cell of a peripheral nervous system, and a precursor cell of any of these cells. The neural cell may be an isolated neural cell, or may be a neural cell present in a body of a human or a non-human animal.

According to the method for cell activation, it is possible to activate an intracellular signaling pathway downstream of Trk without administration of a neurotrophic factor. As a result of the cell activation, a high neuroprotection effect and a high axon regeneration effect can be exhibited. Thus, the method for cell activation can be used for treatment and/or prevention of a nerve-related disease.

An embodiment of the present invention further provides a neural cell into which a nucleic acid construct described in the section [1. Nucleic acid construct] has been introduced in an expressible manner, a non-human animal having the neural cell, and the like.

[4. Recap]

As described above, the present invention encompasses the following aspects.

(1) A nucleic acid construct encoding a fusion polypeptide, the fusion polypeptide including: an intracellular domain of Trk; and a membrane localization sequence.

(2) The nucleic acid construct as set forth in (1), wherein the intracellular domain of Trk is one selected from:

1) a polypeptide having an amino acid sequence represented by any one of SEQ ID NO. 2, 6, 9, 12, or 14; and
2) a polypeptide having an amino acid sequence having a sequence identity of 90% or more with respect to an amino acid sequence represented by any one of SEQ ID NO. 2, 6, 9, 12, or 14.

(3) The nucleic acid construct as set forth in (1) or (2), wherein the membrane localization sequence induces lipid modification of an amino acid.

(4) The nucleic acid construct as set forth in (3), wherein the membrane localization sequence is a farnesylation sequence.

(5) The nucleic acid construct as set forth in any one of (1) to (4), being a vector.

(6) The nucleic acid construct as set forth in (5), being an adeno-associated virus vector.

(7) A pharmaceutical composition containing a nucleic acid construct recited in any one of (1) to (6).

(8) The pharmaceutical composition as set forth in (7), being a drug for a nerve-related disease.

(9) The pharmaceutical composition as set forth in (8), being for neuroprotection or neuroregeneration.

(10) A neural cell into which a nucleic acid construct recited in (1) has been introduced in an expressible manner.

(11) A method for cell activation, including the step of causing an intracellular domain of Trk to be localized at a cell membrane via a membrane localization sequence in a cell.

(12) The method as set forth in (11), wherein the step is carried out by causing one selected from a nucleic acid construct recited in any one of (1) to (6) and a pharmaceutical composition recited in any one of (7) to (9) to be expressed in a neural cell.

(13) A fusion polypeptide including: an intracellular domain of Trk; and a membrane localization sequence. The fusion polypeptide may be encoded by a nucleic acid construct recited in any one of (1) to (4).

The following description will more specifically discuss an embodiment of the present invention with reference to Examples. It is a matter of course that the present invention is not limited to the Examples below and that details of the present invention can have various aspects. Further, the present invention is not limited to the embodiments, but can be altered by a skilled person in the art within the scope of the claims. The present invention also encompasses, in its technical scope, any embodiment derived by combining technical means disclosed in differing embodiments. All literatures described in this specification are incorporated herein by reference.

### EXAMPLES

### [Example 1] Preparation of Constitutive Active TrkB (Hereinafter, CA-TrkB) Vector

(Method)

An AAV vector (referred to as “AAV-CA-TrkB”) expressing an intracellular domain of a TrkB molecule was prepared in the following manner. As an AAV vector in which to incorporate a gene fragment encoding CA-TrkB, used was pAAV-CAG-shuttle-WPRE manufactured by Applied Viromics. A DNA fragment encoding a protein having a myc-tag added to the N-terminus of an intracellular domain of TrkB and a farnesylation signal sequence added to the C-terminus of the intracellular domain was prepared as a gene fragment encoding CA-TrkB (see also FIG. 1). That is, CA-TrkB was provided by: using only the intracellular domain of TrkB without including neither an extracellular domain of TrkB nor a transmembrane domain; and adding the myc-tag and the farnesylation signal sequence to the intracellular domain.

The farnesylation signal sequence (having a base sequence represented by SEQ ID NO: 1) is a signal sequence for causing a protein to be localized at a membrane. The amino acid sequence of the intracellular domain of TrkB is represented by SEQ ID NO: 2, and the base sequence of a gene (DNA) encoding the intracellular domain of TrkB is represented by SEQ ID NO: 3. The base sequence of the gene fragment (the full length of the gene fragment inserted into the AAV vector) encoding CA-TrkB is represented by SEQ ID NO: 4.

AAV-CA-TrkB was designed with an aim to achieve high-level expression, by using a CAG promoter as an expression regulatory system for the gene fragment encoding CA-TrkB and further fusing a WPRE sequence and a poly-A sequence derived from human growth hormone together (see FIG. 2). The WPRE sequence and the poly-A sequence derived from human growth hormone were provided downstream of the farnesylation signal sequence in the AAV vector. The CAG promoter, the WPRE sequence, and the poly-A sequence derived from human growth hormone had been incorporated in the original AAV vector from the start.

Subsequently, the obtained AAV-CA-TrkB was transfected into Cos7 cells, and evaluation of the expression level of AAV-CA-TrkB and activity was carried out.

(Results)

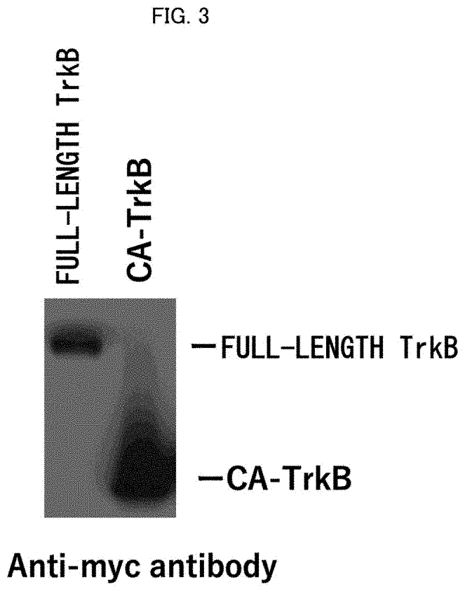

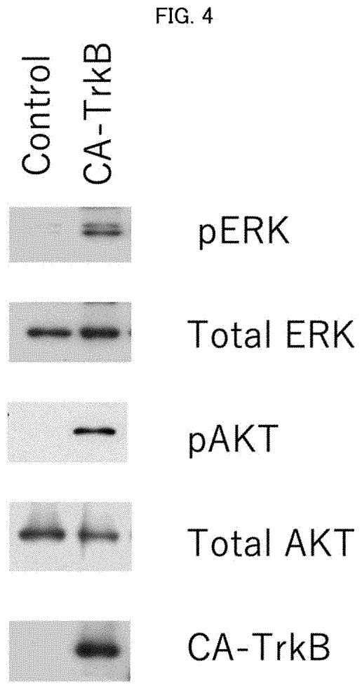

The results of transfecting AAV-CA-TrkB into the Cos7 cells are shown in FIGS. 3 and 4. FIG. 3 shows results obtained from transfecting AAV-CA-TrkB into the Cos7 cells, culturing the cells for 24 hours, and then dissolving the cells and conducting Western blotting with use of an anti-myc antibody (c-Myc antibody (9E10) manufactured by Santa Cruz). It was found that AAV-CA-TrkB had a clearly increased expression level in comparison with a full-length TrkB vector which was subjected to a similar experiment. Note that the full-length TrkB vector is an adeno-associated virus vector in which, in contrast to the structure of AAV-CA-TrkB, a gene fragment encoding full-length TrkB is inserted instead of a gene fragment encoding CA-TrkB.

FIG. 4 shows results obtained from transfecting AAV-CA-TrkB into Cos7 cells, culturing the cells for 24 hours, and then dissolving the cells and conducting Western blotting with use of an anti-pERK antibody (Phospho-p44/42 (Erk1/2) (Thr202/Thr204) manufactured by Cell Signaling Technology), an anti-pAKT antibody (Phospho-Akt (Ser473) (587F11) manufactured by Cell Signaling Technology), an anti-ERK antibody (p44/42 MAPK (Erk1/2) (137F5) manufactured by Cell Signaling Technology), and an anti-AKT antibody (Akt antibody manufactured by Cell Signaling Technology). In the Cos7 cells in which AAV-CA-TrkB was expressed, increased production levels of pERK and pAKT were observed, and it was thus indicated that both signals were activated. However, no changes were observed in total amounts of ERK and AKT. Note that pERK and pAKT are intracellular signals provided downstream of TrkB. A control in FIG. 4 indicates results of a similar experiment conducted with use of an empty vector (a vector obtained by removing the CA-TrkB sequence from the AAV-CA-TrkB vector), and the results show that pERK and pAKT were hardly produced.

### [Example 2] Pathological Analysis of Optic Nerve-Crushed Model

(Method)

—Evaluation Method of Neuroprotection Effect—

To mice (line name: C57BL/6, obtained from Charles River), 2 μL of AAV-DsRed (control group) or AAV-CA-TrkB was administered into an eyeball 14 days before crushing an optic nerve. AAV-DsRed is an adeno-associated virus vector in which, in contrast to the structure of AAV-CA-TrkB, a gene fragment encoding fluorescent protein DsRed is inserted instead of a gene fragment encoding CA-TrkB.

Subsequently, an optic nerve of each of the mice was exposed, and the optic nerve was physically crushed in a portion approximately 1 mm from the backside of the eyeball (see also Non-patent Literatures 6 and 7 for details of the method). Then, 14 days after the crushing of the optic nerve, an expanded specimen of a retina was prepared, and the number of remaining retinal ganglion cells was quantitatively counted. Note that 10 days before the optic nerve was collected (i.e., 10 days before the expanded specimen of the retina was prepared), a dye Fluoro-Gold (the product name of a product manufactured by Fluorochrome) had been injected from the superior colliculus of the brain to retrograde label retinal ganglion cells.

—Evaluation Method of Optic Nerve Regeneration—

To mice (line name: C57BL/6, obtained from Charles River), 2 μL of AAV-DsRed (control group) or AAV-CA-TrkB was administered into an eyeball 14 days before crushing an optic nerve.

Crushing of the optic nerve was carried out similarly as discussed in “Evaluation method of neuroprotection effect” above. Then, 14 days after the crushing of the optic nerve, each mouse was subjected to perfusion fixation, a pathological specimen of the optic nerve was prepared, and the amount of axon regeneration from the part at which the optic nerve had been crushed was quantified. Note that 3 days before the optic nerve was collected (i.e., 3 days before the pathological specimen of the optic nerve was prepared), a fluorescent dye CTB had been injected from the eyeball to color regenerated axonal fibers.

(Results)

—Neuroprotection Effect—

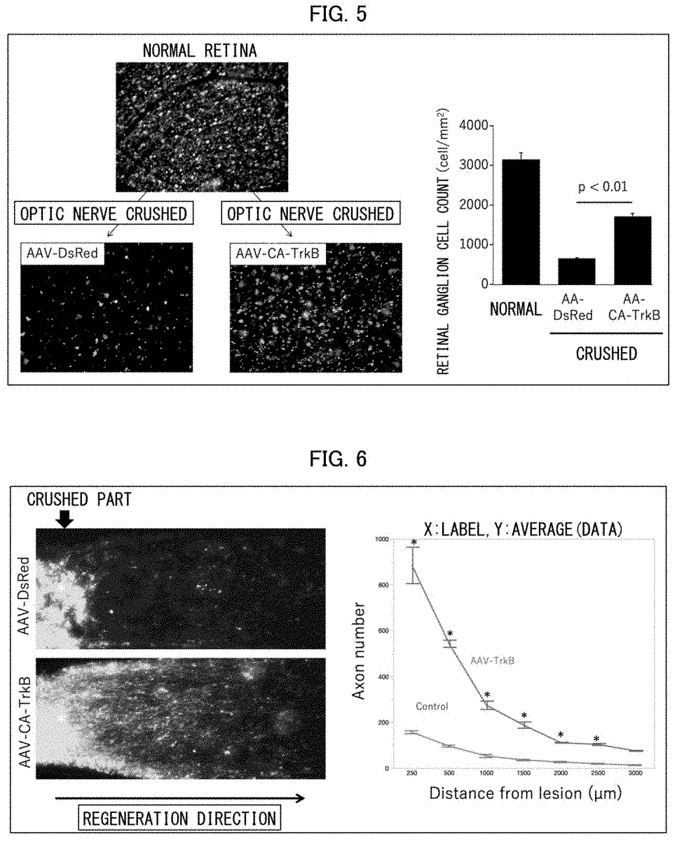

Fourteen days after the crushing of the optic nerve, the number of remaining retinal ganglion cells was quantified. A significant increase was observed in the number of remaining retinal ganglion cells in the group to which AAV-CA-TrkB had been administered (1720±72 cells/mm²), as compared with the group to which AAV-DsRed had been administered (664±14 cells/mm²) (FIG. 5). Note that p<0.01 (one-tailed Student's t test).

—Optic Nerve Regeneration—

Fourteen days after the optic nerve was crushed, the regenerated axons labeled with CTB were measured. The measurement indicated that the group to which AAV-CA-TrkB had been administered had a significant improvement both in the number of regenerated axons and the length of axonal outgrowth, as compared with the group to which AAV-DsRed had been administered (FIG. 6). The X-axis and the Y-axis of the graph in FIG. 6 respectively indicate a distance (μm) from the part at which the optic nerve had been crushed and the number of axons. Note that p<0.05 (one-tailed Student's t test).

### [Example 3] Retinal Ganglion Cell Protection Effect by CA-TrkB (Glaucoma Model Mice)

(Method)

A GLAST knockout mouse, which is a glaucoma model mouse (Reference research paper: Harada T, et al. The potential role of glutamate transporters in the pathogenesis of normal tension glaucoma. The Journal of Clinical Investigation 117: 1763-1770, 2007.), loses retinal ganglion cells to experience decreased visual performance at the age of 3 weeks to 5 weeks. To 10-week-old GLAST knockout mice, 1 μL of AAV-DsRed (control group) or AAV-CA-TrkB was administered into an eyeball (see also Example 2). Then, an expanded specimen of a retina of these knockout mice was prepared when the mice had become 12 weeks old, and the number of remaining retinal ganglion cells was quantitatively counted. To detect the retinal ganglion cells, immunostaining using an RBPMS antibody (an anti-RBPMS antibody manufactured by Merck Millipore) was employed. RBPMS is a marker for retinal ganglion cells.

(Results)

As shown in FIG. 7, a significant increase was observed in the number of remaining retinal ganglion cells in the group to which AAV-CA-TrkB had been administered, as compared with the control group. Note that p<0.05 (one-tailed Student's t test).

### Example 4

(Method)

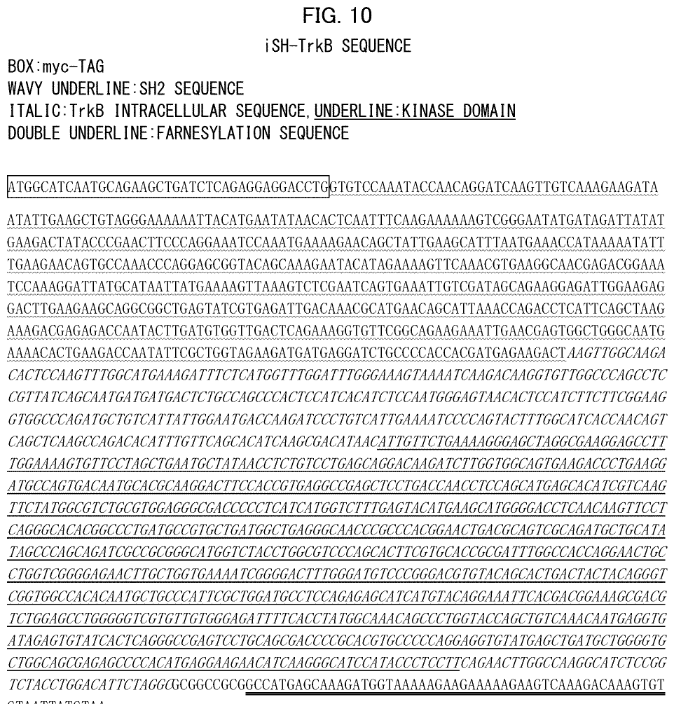

Next, the following vectors expressing a mutant (see also FIG. 8) of CA-TrkB were used to examine signaling activity.
A “Cyto-TrkB” vector: a vector obtained by incorporating, into the AAV vector of Example 1, a DNA fragment encoding a protein including an intracellular domain of TrkB and a myc-tag added to the N-terminus side of the intracellular domain. The “Cyto-TrkB” vector differs from AAV-CA-TrkB of Example 1 only in that no farnesylation signal sequence is added to the C-terminus side of the intracellular domain of TrkB.
A “KD-TrkB” vector: differing from AAV-CA-TrkB of Example only in that an encoded protein includes, in an intracellular domain of TrkB, one amino acid mutation (a lysine to asparagine mutation at the 134th amino acid) that causes a loss of the kinase activity of the intracellular domain.
An “iSH-TrkB” vector: differing from AAV-CA-TrkB of Example 1 only in that an encoded protein further includes an SH2 sequence of p85 inserted between the N-terminus of an intracellular domain of TrkB and a myc-tag. Note that the SH2 sequence is a sequence necessary for forming a complex with a constituent factor p110 of PI3K. The base sequence of a gene fragment (the full length of a gene fragment inserted into the AAV vector) encoding iSH-TrkB is represented by SEQ ID NO: 5 and illustrated as FIG. 10.

Subsequently, in a similar manner to Example 1, these vectors were transfected into Cos7 cells, the cells were dissolved after 24 hours of culturing, and Western blotting was conducted to examine effect on activation of ERK and AKT.

(Results)

As shown in FIG. 8, in the cases where the “Cyto-TrkB” vector and the “KD-TrkB” vector were transfected, no increases of pERK and pAKT were observed, and no activations of pERK signaling and pAKT signaling were thus indicated. In contrast, in the case where the “iSH-TrkB” vector was transfected, a significant increase in AKT activity was indicated in comparison with the case where AAV-CA-TrkB was transfected (in FIG. 8, indicated as “CA-TrkB”). Note that a control in FIG. 8 is the same as the control in Example 1.

### [Example 5] Preparation of Constitutive Active TrkA Vector and Constitutive Active TrkC Vector

(Method)

An AAV vector (referred to as “AAV-CA-TrkA”) expressing an intracellular domain of a TrkA molecule was prepared in accordance with the method of Example 1. That is, AAV-CA-TrkA was prepared in accordance with the method described in Example 1 with use of a fragment having the same structure as the gene fragment encoding CA-TrkB (see Example 1) except that an intracellular domain of TrkA is encoded instead of an intracellular domain of TrkB (see also FIG. 9).

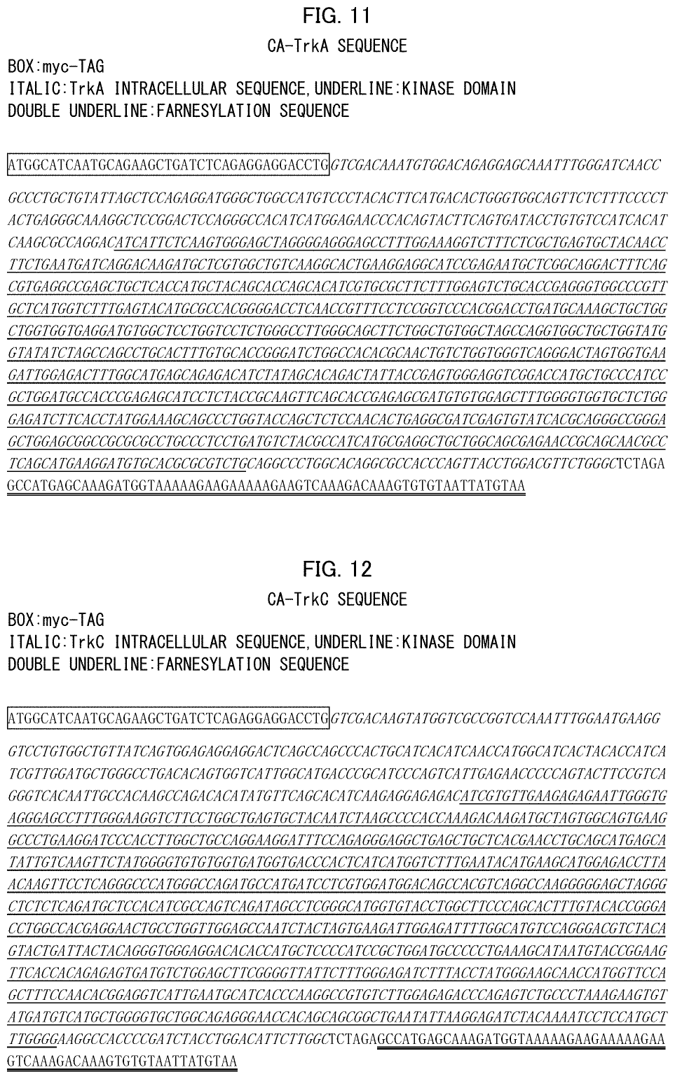

The amino acid sequence of the intracellular domain of TrkA is represented by SEQ ID NO: 6, and the base sequence of a gene (DNA) encoding the intracellular domain of TrkA is represented by SEQ ID NO: 7. The base sequence of the gene fragment (the full length of the gene fragment inserted into the AAV vector) encoding CA-TrkA is represented by SEQ ID NO: 8 and illustrated as FIG. 11.

Similarly, an AAV vector (referred to as “AAV-CA-TrkC”) expressing an intracellular domain of a TrkC molecule was prepared in accordance with the method of Example 1. That is, AAV-CA-TrkC was prepared in accordance with the method described in Example 1 with use of a fragment having the same structure as the gene fragment encoding CA-TrkB (see Example 1) except that an intracellular domain of TrkC is encoded instead of an intracellular domain of TrkB (see also FIG. 9).

The amino acid sequence of the intracellular domain of TrkC is represented by SEQ ID NO: 9, and the base sequence of a gene (DNA) encoding the intracellular domain of TrkC is represented by SEQ ID NO: 10. The base sequence of the gene fragment (the full length of the gene fragment inserted into the AAV vector) encoding CA-TrkC is represented by SEQ ID NO: 11 and illustrated as FIG. 12.

Subsequently, in a similar manner to Example 1, these vectors were transfected into Cos7 cells, the cells were dissolved after 24 hours of culturing, and Western blotting was conducted to examine effect of TrkA and TrkC, which are homologues of TrkB, on activation of ERK and AKT.

(Results)

As shown in FIG. 9, in the cases where AAV-CA-TrkA and AAV-CA-TrkC were transfected, increases of pERK and pAKT were observed, and it was thus indicated that both pERK signaling and pAKT signaling were activated. Note that a control in FIG. 9 is the same as the control in Example 1.

### [Example 6] Visual Performance Recovery Effect by CA-TrkB (Optic Nerve-Crushed Model Mice)

(Method)

From mature mice, a group to which AAV-CA-TrkB was administered was obtained in accordance with the method in Example 2. Fourteen days later, an optic nerve was crushed. Fourteen days and 28 days after the crushing, an electrode port was operatively attached to the superior colliculus of the brain, and a flash-visual evoked potential (F-VEP) was measured.

(Results)

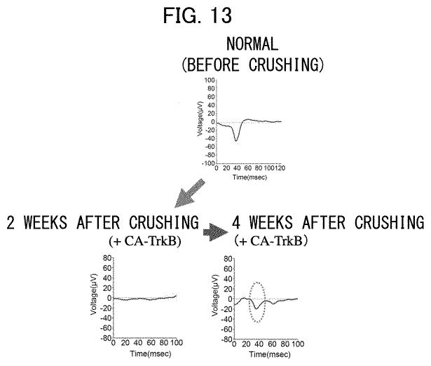

As shown in FIG. 13, reaction of the visual evoked potential was lost 2 weeks after the crushing, but generation of the visual evoked potential was observed 4 weeks after the crushing. It was thus confirmed that the visual performance had partially recovered.

### INDUSTRIAL APPLICABILITY

The present invention can be used for medical purposes involving, for example, nerve-related diseases and the like.

1. A nucleic acid construct encoding a fusion polypeptide, the fusion polypeptide including: an intracellular domain of Trk; and a membrane localization sequence.

2. The nucleic acid construct as set forth in claim 1, wherein the intracellular domain of Trk is one selected from:
1) a polypeptide having an amino acid sequence represented by any one of SEQ ID NO. 2, 6, 9, 12, or 14; and
2) a polypeptide having an amino acid sequence having a sequence identity of 90% or more with respect to an amino acid sequence represented by any one of SEQ ID NO. 2, 6, 9, 12, or 14.

3. The nucleic acid construct as set forth in claim 1, wherein the membrane localization sequence induces lipid modification of an amino acid.

4. The nucleic acid construct as set forth in claim 3, wherein the membrane localization sequence is a farnesylation sequence.

5. The nucleic acid construct as set forth in claim 1, being a vector.

6. The nucleic acid construct as set forth in claim 5, being an adeno-associated virus vector.

7. A pharmaceutical composition comprising a nucleic acid construct recited in claim 1.

8. The pharmaceutical composition as set forth in claim 7, being a drug for a nerve-related disease.

9. The pharmaceutical composition as set forth in claim 8, being for neuroprotection or neuroregeneration.

10. A neural cell into which a nucleic acid construct recited in claim 1 has been introduced in an expressible manner.

11. A method for cell activation, comprising the step of causing an intracellular domain of Trk to be localized at a cell membrane via a membrane localization sequence in a cell.

12. The method as set forth in claim 11, wherein the step is carried out by causing one selected from (i) a nucleic acid construct encoding a fusion polypeptide, the fusion polypeptide including: an intracellular domain of Trk; and a membrane localization sequence and (ii) a pharmaceutical composition comprising the nucleic acid construct to be expressed in a neural cell.

13. A fusion polypeptide comprising: an intracellular domain of Trk; and a membrane localization sequence.
